(19) United States
(12) Patent Application Publication
(10) Pub. No.: US 2026/0026731 A1
(43) Pub. Date: Jan. 29, 2026

(54) WIRELESS ELECTROCARDIOGRAM (ECG) SYSTEM FOR MEDICAL DIAGNOSTICS

(71) Applicant: Gang Yao, Houston, TX (US)

(72) Inventor: Gang Yao, Houston, TX (US)

(21) Appl. No.: 19/075,915

(22) Filed: Mar. 11, 2025

(30) Foreign Application Priority Data
Jul. 24, 2024 (CN) 202410997848.X
Jul. 24, 2024 (CN) 202421761733.2

(51) Int. Cl. A61B 5/319; A61B 5/00; A61B 5/263; H04M 1/72412

(52) U.S. Cl. CPC A61B 5/319; A61B 5/0006; A61B 5/0022; A61B 5/263; A61B 2560/0223; A61B 2562/222; H04M 1/72412

(57) ABSTRACT

This invention relates to a wireless electrocardiogram (ECG) system includes a smart terminal and a plurality of wearable wireless lead front ends applying advanced short-range wireless communication technology for bidirectional information transmission, configured such that when in the simulation testing mode, the smart terminal sends a calibrated/quantized ECG signal to each lead front end as an input reference signal, the lead front end detects the reference signal and uploads it to the smart terminal, the smart terminal compares the reference signal measured by each front end with the original signal and generates a response curve; when in the actual measurement mode, the body surface electrodes collect the ECG/biopotential signals, which are pre-processed by each lead front end and wirelessly uploaded to the smart terminal, the smart terminal corrects the ECG data of each lead based on the response curve, obtains an ECG report with diagnostic accuracy and outputs it.

### CROSS-REFERENCE TO RELATED PATENT APPLICATIONS

This application claims priority to and the benefit of Chinese Patent Application Nos. 202410997848.X and 202421761733.2, both filed Jul. 24, 2024, which are incorporated herein in their entireties by reference.

### FIELD OF THE INVENTION

The invention generally relates to medical devices, and more particularly to a wireless electrocardiogram system for diagnosis.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Cardiovascular diseases are the leading cause of death globally. Cardiovascular diseases are a group of disorders of the heart and blood vessels and include coronary heart disease, cerebrovascular disease, rheumatic heart disease and other conditions. In the diagnosis and treatment of cardiovascular diseases, electrocardiographs play a very important role. Currently, among medical-grade electrocardiographs, conventional multi-lead hospital electrocardiographs that meet the diagnostic accuracy performance indicators are widely used. However, these medical-grade electrocardiographs cannot simultaneously meet the requirements of conventional multi-lead (more than 10 leads) electrocardiographs for medical diagnostic purposes that meet the national diagnostic accuracy standards and are wearable without lead wires.

Therefore, a heretofore unaddressed need exists in the art to address the aforementioned deficiencies and inadequacies.

### SUMMARY OF THE INVENTION

In view of the deficiencies and inadequacies in the art, this invention provides a wearable multi-lead electrocardiogram (ECG) system without lead wires that utilizes a new generation of wireless short-range communication technology to meet the requirements for the accuracy performance of medical diagnostic ECG equipment and adopts closed-loop adaptive control, which the measurement accuracy, the wearability and convenience of use can be greatly improved, and the number of leads meets the conventional multi-lead requirements.

In one aspect, the invention relates to a wireless ECG system for diagnosis comprising a smart terminal; a plurality of lead front ends, each lead front end being coupled with a corresponding body surface electrode configured to be attached to a body? of a subject for collecting ECG/biopotential signals, the number of the lead front ends being seven or more, or ten or more; and a bidirectional wireless communication channel established between each lead front end and the smart terminal for independent bidirectional information transmission. The wireless ECG system is configured to have a simulation testing mode and an actual measurement mode, such that close-loop adaptive ECG information collection and process are achieved by switching between the simulation testing mode and the actual measurement mode online in real time.

When in the simulation testing? mode, the smart terminal first acts as a signal source to synchronously send a calibration/digitalized ECG signal as an input reference signal to each lead front end through the bidirectional wireless communication channel. and the information obtained by the lead front end for detecting the reference signal is transmitted back to the smart terminal through the bidirectional wireless communication channel, and the smart terminal then compares and calibrates the reference signal detected by each lead front end with the calibration/digitalized ECG signal originally sent and generates the test response curve of each channel.

When in the actual measurement mode, the ECG/biopotential signal collected by each body surface electrode is pre-processed by the lead front end connected to one by one, and then uploaded to the smart terminal through the bidirectional wireless communication channel, and the smart terminal corrects the ECG data actually measured by each lead in real time based on the response curve to obtain and output the ECG report that meets the national standards for high medical diagnostic accuracy.

In one embodiment, each lead front end comprises a first NearLink module, and the smart terminal comprises a second NearLink module; the first StarFlash NearLink module and the second NearLink module perform short-distance wireless communications through the NearLink mode, and the bidirectional information transmission is realized between each lead front end and the smart terminal through the first NearLink module and the second NearLink module, respectively.

In one embodiment, the smart terminal comprises a signal generation module and a data processing module, the signal generation module is configured to generate a calibration/digitalized ECG signal and an adaptive control signal, and transmit it downlink to each lead front end as an input reference signal; the data processing module is configured to compare the information detected by the reference signal wirelessly uplinked back from each lead front end with the original transmitted signal and generate a test response curve of each lead front end; and the measured ECG data of each lead is corrected in real time according to the response curve, thereby realizing the close-loop adaptive acquisition and processing of multi-lead ECG signals without lead wires.

In one embodiment, the smart terminal comprises a time base and control module, the time base and control module is configured to generate a time base signal and synchronously control the signal sampling and information wireless transmission of each lead front end in a concurrent manner, and the internal calibration of each lead front end and the input switching of the ECG measurement between the simulation testing mode and the actual measurement mode.

In one embodiment, the smart terminal comprises a storage and output module, the storage and output module are connectable to the cloud through the Internet and is configured to cache and normalize the ECG data and then store it locally and/or upload it to the cloud, and output the ECG data in the form of an ECG report.

In one embodiment, each lead front end comprises an input circuit module and a signal processing and control module, the input circuit module pre-processes the ECG/biopotential signals collected by the body surface electrodes, and the signal processing and control module uploads the pre-processed ECG/biopotential signals through the wireless communication channel under the control of the smart terminal; and completes the input and output of the reference signal for the simulation testing mode transmitted by the smart terminal.

In one embodiment, the wireless ECG system further comprises a storage bin, wherein the storage bin is operably connected to the smart terminal via a C-Type interface and/or NearLink wireless communication channels; the storage bin is configured to store the lead front ends and charge the lead front ends; before each ECG test, the storage bin cooperates with the smart terminal to perform initialization calibration, calibration and display of a charging level of each lead front end.

In one embodiment, the wireless ECG system further comprises one or more expandable lead front end, wherein a bidirectional wireless communication channel is established between the expandable lead front end and the smart terminal, and the expandable lead front end is configured to detect other biopotential information of the subject.

In one embodiment, the communication protocol used by the wireless communication channel comprises NearLink, WiFi, Bluetooth, radio frequency identification (RFID), near field communication (NFC), Zigbee, ultra-wideband (UWB), ANT, Z-Wave, potential sensing communication and infrared (IR), Thread, and any combination thereof.

In one embodiment, each lead front end is a wireless micromation wearable member, and logo and color of the wireless wearable member of each lead front end are determined according to electrode identifier and color code specified by national standards.

In one embodiment, the wireless ECG system further comprises a positioning ruler personalized/customized specifically for the subject, wherein the positioning ruler is formed of an instantly solidified or shape memory medical material on site of the chest of the subject to conform to the curve shape of the chest of the subject.

In one embodiment, the instantly solidified medical material comprises a low-temperature thermoplastic tape, a thermoplastic splinting material, a food-grade dental mold silicone, or other materials that meet human safety standards.

In one embodiment, the positioning ruler has two ends marked with exact positions relative to body markers of the individual subject, and a plurality of holes formed therebetween for placing the body surface electrodes (on the chest of the subject, C1-C6/V1-V6).

In one embodiment, the holes of the positioning ruler is personalized/customized such that when the positioning ruler is placed on the chest of the subject with the ends aligned with the body markers, the positions of the holes coincide respectively with the positions of the body surface electrodes to be placed.

In one embodiment, in operation, the body surface electrodes are placed on the chest at the positions of the holes of the positioning ruler.

According to the invention, the wireless ECG system, which meets the requirements for the accuracy of reconstruction of the input signal of the ECG diagnostic equipment specified in the national standards, can be used as an ECG diagnostic device for routine rest ECG testing, as well as a wireless multi-lead dynamic ECG machine (Holter) or exercise (stress) ECG tester to continuously record the ECG report of the human body in life and exercise for a long time; it can also be used as a wearable portable ECG monitor (Monitor). It is only necessary to wear the corresponding electrodes and the miniaturized lead front end in advance and use them in conjunction with the smart terminal, one can safely and conveniently perform ECG testing, recording, storage, display and transmission via internet/cloud for medical diagnosis at any time, any place and for any duration.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 is a schematic diagram of a diagnostic wireless electrocardiogram system according to one embodiment of the invention.

FIG. 2 is a schematic diagram of a storage bin storing a plurality of lead front ends in a diagnostic wireless electrocardiogram system according to one embodiment of the invention.

FIG. 3 shows schematically a personalized positioning rule placed on the chest of a subject according to one embodiment of the invention.

### DETAILED DESCRIPTION OF THE INVENTION

The invention will now be described more fully hereinafter with reference to the accompanying drawings, in which exemplary embodiments of the invention are shown. However, this invention may be embodied in many different forms and should not be construed as limited to the embodiments set forth herein. Rather, these embodiments are provided so that this specification will be thorough and complete and fully convey the invention's scope to those skilled in the art. Like reference numerals refer to like elements throughout.

The terms used in this specification generally have their ordinary meanings in the art, within the context of the invention, and in the specific context where each term is used. Certain terms used to describe the invention are discussed below, or elsewhere in the specification, to provide additional guidance to the practitioner regarding the description. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, or section without departing from the invention's teachings.

Furthermore, relative terms, such as “lower” or “bottom” and “upper” or “top,” may be used herein to describe one element's relationship to another element as illustrated in the figures. It will be understood that relative terms are intended to encompass different orientations of the wireless ECG system in addition to the orientation depicted in the figures. For example, if the wireless ECG system in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the wireless ECG system in one of the figures is turned over, elements described as “below” or “beneath” other elements would then be oriented “above” the other elements. Therefore, the exemplary terms “below” or “beneath” can encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The term “NearLink” or “NearLink/SparkLink”, used in this specification, refers to a new generation short-range wireless communication technology launched by the NearLink Alliance. In contrast to traditional WiFi and Bluetooth wireless communication technologies, NearLink has undergone extensive innovative upgrades and has incorporated key 5G technologies. It has redefined the standards for wireless connectivity, achieving qualitative leaps in terms of speed, latency, transmission distance, security, and reliability. It can be considered as an upgraded hybrid version of WiFi and Bluetooth.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

An electrocardiogram (ECG or EKG) is a simple, quick test that measures the electrical activity of the heart. It is configured to diagnose and monitor heart conditions such as heart attacks, arrhythmias, poor blood supply, heart inflammation, cardiac arrest, and more, and can also detect non-heart conditions such as electrolyte imbalances and lung diseases. It can also be used for evaluating medication effectiveness, preoperative assessment, and screening for individuals in high-risk occupations and those participating in sports.

This invention discloses a wireless ECG system including a smart terminal and multiple micromation wearable wireless lead front ends. When in the simulation testing mode, the smart terminal sends a calibration/digitized ECG signal to each lead front end as an input reference signal. The lead front end detects the reference signal and uploads it to the smart terminal. The smart terminal compares the reference signal measured by each front end with the original signal and generates a response curve. When in the actual measurement mode, the body surface electrodes collect the ECG signals, which are pre-processed by each lead front end and wirelessly uploaded to the smart terminal. The smart terminal corrects the ECG data of each lead based on the response curve to obtain an ECG report with diagnostic accuracy and outputs it. The technical solution of the invention can realize resting ECG measurement without lead wires with medical diagnosis-level accuracy. It can also be used as a dynamic ECG or exercise ECG tester for long-term continuous recording of ECG data of human body in life and exercise.

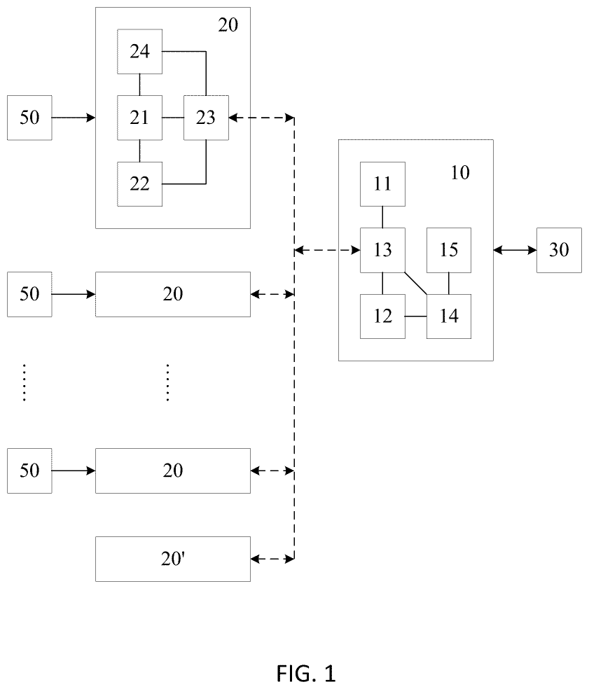

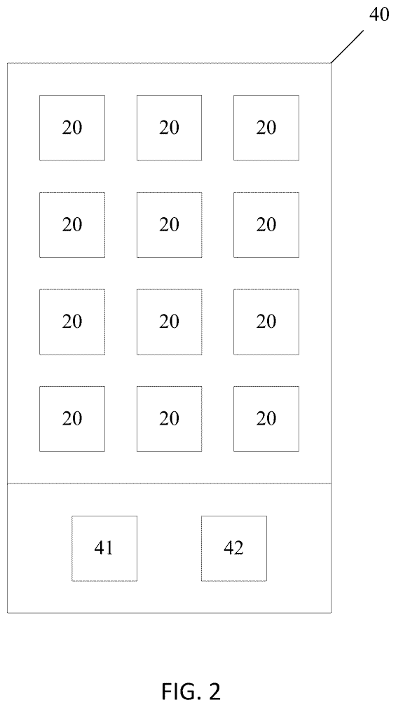

Referring to FIGS. 1 and 2, an embodiment of the wireless ECG system is shown. The wireless ECG system includes multiple body surface electrodes 50, multiple lead front ends 20 coupled with the multiple body surface electrodes 50, multiple expandable lead front ends 20′, a smart terminal 10 coupled with the multiple lead front ends 20 and the multiple expandable lead front ends 20′, an output device 30 coupled with the smart terminal 10, and a storage bin 40. In one embodiment, a wireless communication channel capable of bidirectional information transmission is established between each of the multiple lead front ends 20 and the smart terminal 10, and each body surface electrode 50 is connected to a corresponding lead front end 20. The wireless ECG system is configured to operate cyclically in a simulation testing mode and an actual measurement mode under the control of the smart terminal 10. The close-loop adaptive ECG information collection and processing of the wireless ECG system is realized by switching the simulation testing mode and the actual measurement mode online in real time.

When the wireless ECG system is in the simulation testing mode under the control of the smart terminal 10, the smart terminal 10 first acts as a signal source to synchronously send a calibration/digitized electrocardiogram signal to each lead front end 20 through its wireless communication channel as a system input reference signal. Each lead front end 20 transmits the information obtained by detecting the reference signal back to the smart terminal 10 through its own individual? wireless communication channel. The smart terminal 10 then compares and calibrates the reference signal detected by each lead front end 20 with the original transmitted signal and generates a test response curve for the channel.

When the wireless ECG system is in the actual measurement mode under the control of the smart terminal 10, the electrocardiogram signals collected by each body surface electrode 50 are pre-processed by each lead front end 20 and uploaded to the smart terminal 10 through their own independent wireless communication channels. The smart terminal 10 corrects and normalizes the electrocardiogram data actually measured by each lead front end 20 based on the response curve in real time and obtains and outputs the ECG report. The calibration signal and the digitized ECG for analysis can be generated using, but not limited to, the international (CTS/CSE) database recommended by the international standard IEC 60601-2-25 “Particular requirements for the basic safety and essential performance of electrocardiographs”.

In one embodiment, each body surface electrode 50 or other medical biopotential parameter sensor fixed to a specific location of the human body transmits the detected medical biopotential signal of the patient to the corresponding lead front end 20. Because the lead front end 20 is powered by an internal micro-rechargeable battery and there is no lead wire connected to other parts of the system during ECG (electrocardiogram) measurement, the safety and fault tolerance of operation are greatly improved.

In one embodiment, each lead front end 20 is designed as a wireless micrimation wearable member with wearable and wireless communication functions. The wearable member is a miniaturized component with no electrode lead wires. that can be connected to the corresponding body surface electrode 50, which can be convenient for patients to wear it comfortably for a long time. In one embodiment, the electrode identifier and color code of each lead front end 20 are determined according to the electrode connection mark and color code specified by the national standard. The lead front ends 20 with different colors and codes are placed in the corresponding positions in the storage bin 40 according to the order in which they are connected to the body surface electrodes 50.

In one embodiment, the number of lead front ends 20 includes at least 7 Frank leads or conventional 12 leads. To meet the data transmission needs of Frank leads or conventional 12-lead ECG and other extended medical biopotential information, at least 10 or more bidirectional transmission wireless communication channels are included between each lead front end 20 and the smart terminal 10. Scalability means that the medical biopotential parameter information such as breathing, blood oxygen saturation, blood pressure, body temperature, etc. collected synchronously by other medical physiological sensors and wireless stethoscopes can be wirelessly transmitted to the smart terminal 10 by the expandable lead front end 20′ for comprehensive processing. The use of NearLink technology can expand the number of wireless communication channels to 256. The lead front end 20 and the smart terminal 10, and the expandable lead front end 20′ and the smart terminal 10 can be connected through corresponding wireless communication channels and carry out bidirectional information transmission.

Wireless communication methods include, but are not limited to, NearLink (NearLink/SparkLink), WiFi®, Bluetooth®, Radio Frequency Identification (RFID), Near Field Communication (NFC), ZigBee, Ultra-Wideband (UWB), Electric Potential Sensing Communication (EPSComm), ANT, Z-Wave, Thread and infrared technology.

In one embodiment, the NearLink technology is configured to achieve short-range wireless communications. The time-division multiplexing (TDM) method used by the NearLink technology can ensure that at most 256 bidirectional wireless communication channels can be established simultaneously between each lead front end 20 and the expandable lead front end 20′ and the smart terminal 10, and only ten to twenty wireless communication channels are required in the wireless ECG system of this embodiment to meet all information transmission requirements.

As shown in FIG. 1, in the exemplary embodiment, the lead front end 20 includes an input circuit module 21, a signal processing and control module 22, a first NearLink module 23 and a power supply module 24, each of which is composed of existing miniaturized integrated circuit devices that have been verified for practical use, with unique functions, safety and reliability. The input circuit module 21 operably preprocesses the ECG/biopotential signals collected by the body surface electrode 50. The signal processing and control module 22 is configured to switch between the simulation testing mode and the actual measurement mode under the control of the smart terminal 10, such that when in the actual measurement mode, the pre-processed analog ECG/biopotential signal is converted into digital under the control of the smart terminal 10 and uploaded to the smart terminal 10 by the first NearLink module 23 via the wireless communication channel; when in the simulation testing mode, the first NearLink module 23 receives the simulation detection reference signal transmitted by the smart terminal 10 via the wireless communication channel and sends it to the signal processing and control module 22 for digital-to-analog conversion and sends it to the input end of the lead front end 20; and the output signal of the lead front end 20 is uploaded to the smart terminal 10 by the first NearLink module 23 via the wireless communication channel. The power supply module 24 is configured to supply power to the input circuit module 21, the signal processing and control module 22 and the first NearLink module 23. In some embodiments, all the above modules are composed of commercially available miniaturized integrated circuit chips, and thus their performances are safe and reliable. Among them, the input circuit module 21 adopts a high-precision analog front end (AFE), and the signal processing and control module 22 adopts a digital signal processing element such as digital signal processing/controlling (DSP/C) element. Each device in the lead front end 20 adopts system-in-a-package (SIP) packaging (system-level packaging) to make it as miniaturized and lightweight as possible, so that users can wear it comfortably for a long time. When measuring the electrocardiogram, the user only needs to equip or rent a set of lead front ends 20 placed in the storage bin 40 and connect each lead front end 20 to each surface electrode 50 fixed on the body at a certain position according to the doctor's instructions. Combined with the smart terminal 10 with the NearLink function, following the App screen prompts of the smart terminal 10, the multi-lead real-time ECG signal wireless close-loop adaptive control can be automatically/manually collected and processed. Normally, one only need to wear the miniaturized, lightweight, lead-free multiple lead front ends 20 and the body surface electrodes 50 connected thereto, and all other calibration, control, recording, storage, data acquisition and processing functions are completed by the wirelessly connected smart terminal 10. The smart terminal 10 can be a smart phone or tablet, a notebook, or a desktop computer with NearLink wireless communication function and equipped with corresponding application software App. The use of the ECG device can not only greatly improve the connection of electrode lead wires and the inconvenience of wearing multi-lead devices in various existing medical diagnostic electrocardiographs, but also reduce the cost of separately configuring dedicated electrocardiographs for users who already have universal smartphones, tablets, and laptops with NearLink functions.

In one embodiment of the device as shown in FIG. 1, the smart terminal 10 includes a signal generation module 11, a data processing module 12, a second NearLink module 13, a time base and control module 14, and a storage and output module 15. The signal generation module 11 is configured to generate a calibration/quantization ECG signal and an adaptive control signal. The data processing module 12 is configured to feed back the calibration/quantization ECG signal and the adaptive control signal to each lead front end 20 in real time through the second NearLink module 13, receive and generate a test response curve of each lead front end 20; and perform real-time correction on the measured electrocardiogram data of each lead front end 20 according to the response curve, and then perform secondary caching of the electrocardiogram data through the storage module 15. The second NearLink module 13 operably sends the signal of the signal generation module 11 to each lead front end 20 via a wireless communication channel and receives signals and data from each lead front end 20. The time base and control module 14 is configured to generate a high-precision time base signal and synchronously control the signal sampling and transmission of each lead front end 20 in a concurrent manner, as well as the input switching between the simulation testing mode and the actual measurement mode. The storage and output module 15 operably buffers and finally stores the data received by the smart terminal 10, and can be connected to the cloud through the Internet or mobile Internet to normalize the ECG data and then store it locally and/or upload it to the cloud for remote diagnosis and big data analysis, and output the ECG data in the form of an ECG report through the output device 30, which is a high-resolution thermal/laser printer or other general display device.

Between each lead front end 20 and the smart terminal 10, the first NearLink module 23 and the second NearLink module 13 perform short-range wireless communication in the form of NearLink and realize bidirectional data transmission through each first NearLink module 23 and the second NearLink module 13. Since the NearLink technology has high reliability, high bandwidth, high concurrency, multiple connections, low latency, precise synchronization, strong anti-interference and other characteristics in short-range wireless communication compared with other technologies, taking a preferred embodiment of the present application as an example, the NearLink low-power access smart link enhanced (SLE) technology is adopted. This technology can support up to 256 users concurrently accessing data transmission, which is much higher than the upper limit of no more than 20 channels required by this embodiment. In addition, according to the requirements of the signal frequency response of 0.67-150/500 Hz and the sampling rate of not less than 500 times/second stipulated in the international standard, based on the high data transmission rate unique to the NearLink technology, its time-division multiplexing (TDM) method can ensure the bidirectional transmission of high-precision ECG sampling data and real-time calibration and control signals between each lead front end 20 and the smart terminal 10, and can achieve online automatic calibration and data acquisition, transmission and real-time correction under close-loop adaptive control. The preferred implementation method of this embodiment can also find application scenarios for medical equipment for the Internet of Everything (IoE) and NearLink short-range wireless communication technology.

In one embodiment, the output device 30 includes, but is not limited to, a high-resolution (>=2400 DPI) commercial laser printer (or a traditional electrocardiograph recording medium) to print out an electrocardiogram report; a high-resolution (>=3840×2160) display for displaying or monitoring an electrocardiogram or a vector cardiogram (VCG). The smart terminal 10 outputs the electrocardiogram data in the form of a diagnostic electrocardiogram report through the output device 30.

FIG. 2 shows schematically one embodiment of the storage bin 40, which is operably connected to the smart terminal 10 through a NearLink wireless communication channel or via a C-type interface/cable. The storage bin 40 is configured to store multiple lead front ends 20 and charge the lead front ends 20. Before each electrocardiogram test, the storage bin 40 cooperates with the smart terminal 10 to initialize, calibrate and align all the lead front ends 20 and the entire system. When using the storage bin 40, the multiple lead front ends 20 are stored in order. The storage bin 40 is connected to the smart terminal 10 via a C-type interface/cable or other connecting interfaces and a wireless communication channel. Each lead front end 20 stored is charged through a charging module 41 and the charging level of each lead front end 20 is displayed. The storage bin 40 includes a charging module 41 and a NearLink and signal processing module 42. The NearLink and signal processing module 42 completes the calibration and alignment functions of the system before each use in a close-loop automatic manner under the control of the calibration calibration/digitized ECG signal generated by the smart terminal 10 and program instructions and provides charging/charging level display for each lead front end 20. The storage bin 40 can store 12 sets of lead front ends 20. Each lead front end 20 with its own lead color code is placed in the storage bin 40 in order in the corresponding color/serial number jack for standby use. Among them, one or two of the lead front ends 20 are used as backups, ready to replace the low-power lead front ends 20 at any time. When preparing for ECG detection, opening the cover of the storage bin 40 automatically triggers the NearLink and signal processing module 42 to start working under the control of the smart terminal 10, and starts the self-test and calibration test process for each lead front end 20. After each lead front end 20 taken out from the storage bin is connected to each body surface electrode 50, the disconnection detection signal contained in the chip of the input circuit module 21 in the lead front end 20 is uploaded to the smart terminal 10 for display and alarm. The program-controlled amplification function contained in the control module will complete automatic/manual gain control according to the instructions of the smart terminal 10 to ensure a suitable detection dynamic range. For the nonlinear response of the amplitude and frequency of each wireless communication channel obtained by calibration/quantization of the electrocardiogram signal detection, the smart terminal 10 uses digital signal process (DSP) application software functions such as curve fitting, table lookup, and digital filtering to make real-time corrections, and stores the detection data of each lead front end 20 after correction in the secondary cache area, and then stores it in the output data area after normalization, time axis alignment, and other adjustments.

Specifically, when the patient uses the system to measure the electrocardiogram, the following workflow is followed. First, each body surface electrode 50 is fixed to a specific location of the human body and connected to each lead front end 20 one by one. The system automatically powers on and stably establishes a wireless communication channel. After completing the initialization self-check calibration under the control of the smart terminal 10, the ECG signals detected by each body surface electrode 50 are synchronously sampled, pre-processed by each channel input circuit module 21 and converted by the signal processing and control module 22, and then wirelessly sent to the smart terminal 10 through the first NearLink module 23. The independent power supply module 24 included in each lead front end 20 provides power supply and the reference voltage required for analog-to-digital/digital-to-analog (AD/DA) conversion. The smart terminal 10 receives the periodically switched reference signal and the ECG measured signal wirelessly uploaded by each lead front end 20 through the second NearLink module 13. Under the joint action of the data processing module 12 and the time base and control module 14, the data uploaded by each wireless communication channel is stored in the corresponding first-level buffer area, so that the data of each lead front end 20 can be aligned and normalized with the lead time base/time axis in real time.

In the exemplary workflow, the smart terminal 10 periodically switches to the simulation testing mode. The signal generation module 11 generates: online calibration/digitized ECG signals based on the test performance requirements of the international standard IEC 60601-2-25 “Particular requirements for the basic safety and essential performance of electrocardiographs” and the CTS/CSE database recommended, as well as adaptive control signals, and wirelessly sends them downlink to the lead front end 20, and the signal processing and control module 22 performs digital-to-analog conversion to form a detection reference signal and sends it to the input end of the lead front end 20; the lead front end 20 performs analog-to-digital conversion on the detection result of this reference signal, and the first NearLink module 23 transmits it uplink to the smart terminal 10 via the wireless communication channel. The smart terminal 10 compares the received reference signal detection information of each channel with the originally sent calibration/digitized electrocardiogram signal to obtain a response curve including the real-time gain, amplitude frequency response, etc. of the system, and automatically corrects the measured electrocardiogram data in real time according to the response curve to improve accuracy and reduce interference, and then performs secondary cache processing on the obtained electrocardiogram report data. The high-precision time base signal generated by the time base and control module 14 synchronously controls the sampling, calibration and switching of each lead front end 20. Finally, the smart terminal 10 completes the storage and output of the diagnostic electrocardiogram report data that complies with the international medical electrical equipment standard IEC 60601-2-25 “Particular requirements for the basic safety and essential performance of electrocardiographs” through the output device 30.

The wireless ECG system according to the exemplary embodiment of the invention can improve the accuracy of ECG detection, meet the accuracy requirements of the international standard, and meet the standard requirements of ECG equipment for medical diagnosis. Since weak electricity is used to charge the lead front end 20, and it is powered by an internal rechargeable battery when worn and has no physical connection with other parts of the ECG system, the safety of the ECG system is greatly improved. The application scenarios of the ECG system are not limited to hospitals, health centers and clinics. It can be operated and used by patients, family members with no medical professional training at home. With the help of the personalized positioning ruler described below, chest lead electrodes can be accurately placed by non-professionals at the standard positions on the patient's chest, thereby obtaining high-quality ECG recordings. It can detect ECGs in a timely manner and report ECG conditions to doctors or caregivers via internet at any time.

In one aspect, the invention also provides a personalized/customized positioning ruler for patients to place electrodes by themselves. When the patient visits a hospital for the first time, a medical staff determines the exact position of the 6 chest electrodes on the patient's chest and the lead front end 20 connected thereto. Then, a rigid semi-permanent ruler with personalized fixed electrode position holes for the patient's chest leads C1-C6 can be made on site using quick-curing medical polymeric/shape memory material, and the two ends of the personalized positioning ruler can be marked with the exact position relative to the patient's body (such as the position of the two nipple and, if necessary, supplement with the suprasternal fossa or the tip of the xiphoid process as the personalized third positioning point for the ruler. Later, the patient or other non-medical personnel can accurately locate the electrode position of each lead at home according to the relative position of the personalized positioning ruler, thereby obtaining high-quality ECG recordings without the presence of a medical staff in the telemedicine/home medical care scenario.

Without intent to limit the scope of the invention, exemplary embodiments of the personalized/customized positioning rulers are given below.

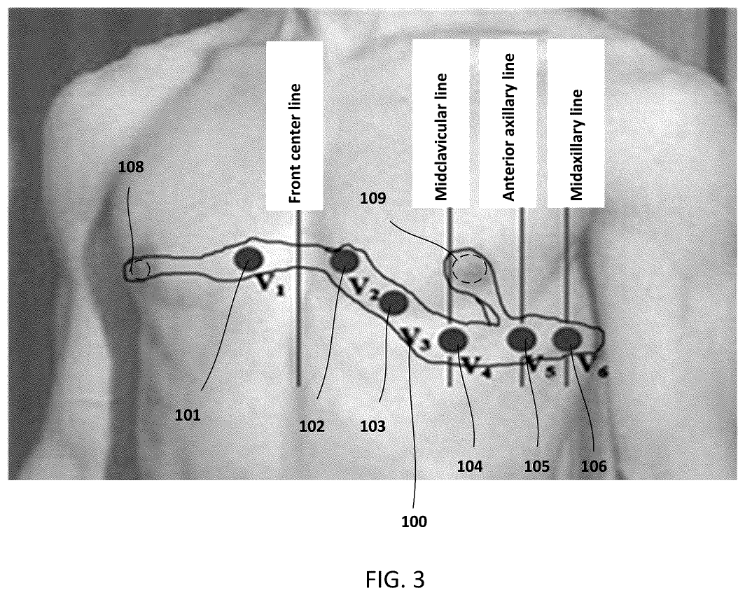

As listed in Table 1 and shown in FIG. 3, in the universal 12-lead ECG for medical diagnosis that meets the international standard, only six chest lead electrode positions need to be accurately located.

TABLE 1
Name and placement of body surface electrodes

| Name of electrodes | Placement of electrodes |
|---|---|
| LA | Left upper limb |
| RA | Right upper limb |
| LL | Left lower limb |
| RL | Right lower limb |
| V1 | 4th intercostal space, right sternum |
| V2 | 4th intercostal space, left sternum |
| V3 | Between leads V2 and V4 |
| V4 | 5th intercostal space, left midclavicular line |
| V5 | On the left axillary line in the 5th intercostal space |
| V6 | 5th intercostal space on the left midaxillary line |

To ensure the safety of use, the positioning ruler is made of a quick-curing polymeric/shape memory material that meets human safety standards. In some embodiments, the low cost quick-curing polymeric/shape memory material includes, but is not limited to, low-temperature thermoplastic tape, thermoplastic splinting materials such as orthopedic medical polymer external fixation and shaping splinting materials, and/or food-grade dental mold silicone such as putty-type hand-kneading material, dental putty, etc.

When the thermoplastic tape or plate is used, a qualified medical technician/staff firstly cuts the thermoplastic tape or plate with a width of about one inch (2.5 cm) into a ruler-shaped strip 100 with a length of about 16 inches (40 cm), as shown FIG. 3, secondly, softens the ruler-shaped strip 100 with an electric hair dryer or immersed in 50-75° C. hot water, and then places the ruler-shaped strip 100 close to the chest of a person being tested to conform to the shape of the chest curve, and bends the ruler-shaped strip 100 along the long axis to cover the C1-C6 position of the ECG chest lead and with its two ends covering the two nipples (and the third positioning point, if necessary) of the person being tested, as shown FIG. 3, and lets the ruler-shaped strip 100 stand for about 3-5 minutes to cool and solidify.

The qualified medical technician/staff then uses a marker corresponding to a color of each lead on the ruler-shaped strip 100 to accurately marks the specific position of each lead electrode on the ruler-shaped strip 100 and the positions of the two ends of the ruler-shaped strip 100 relative to the two nipples of the person being tested. For example, as shown in FIG. 3, marks 101, 102, 103 104, 105 and 106 on the ruler-shaped strip 100 are corresponding to the positions of lead electrodes V1, V2, V3, V4, V5 and V6, respectively, and marks 108 and 109 are corresponding to the positions of the two ends of the ruler-shaped strip 100, respectively. After the ruler-shaped strip 100 is removed from the chest of the person, holes are punched in all the marked positions (e.g., 101, 102, 103 104, 105 and 106) on the positioning ruler and the name of the person being tested is indicated. The personalized customization of the ruler-shaped strip 100, i.e., the personalized positioning ruler, specifically for the person is completed. The personalized positioning ruler (making a backup if necessary) is stored in a box to avoid squeezing or stretching.

When the food-grade dental mold silicone is used, the qualified medical technician/staff first accurately locates the position of each chest lead electrode on the chest of a person being tested and adheres the chest lead electrodes to the chest of the person at their corresponding positions, and then applies a thin layer of electrode gel near the two nipples and the ECG electrode distribution area on the chest, which prevents the silicone from adhering to the skin in the next processes. Two components of the food-grade dental mold silicone (dental putty) are missed to form a putty (soft plasticine) of a hand-kneading material. This toothpaste-like dental putty is then shaped into a long rod for later use.

The soft silicone rod of the putty is placed to the chest of the person to conform to the shape of the chest curve, and kneaded along the long axis to cover all the chest lead electrodes adhered on the chest of the person, with its two ends covering the two nipples of the person, where both ends of the silicone rod have accurate positioning marks with the two nipples of the person. Since the electrodes adhered on the chest of the person have columnar protrusions in the center, the soft silicone rod can form holes around the electrode protrusions when laid at the center of each electrode. After standing and solidifying, the soft silicone rod is gently removed from the chest of the person, each electrode is marked with a corresponding color and the name of the person is indicated. The personalized customization of the positioning ruler specifically for the person is completed.

When the person needs to measure ECG by himself/herself without a medical staff, he/she can press the positioning ruler against the patient chest and align the marks on both ends of the positioning ruler with the two nipples to determine the position of each chest lead easily and accurately.

In sum, the wireless ECG system according to embodiments of the invention uses the smart terminal as the G (management) node of the NearLink technology transmission, and the lead front end and storage bin connected by each wireless communication channel as the managed (T) node to form a communication domain. According to the performance, power consumption and cost requirements of NearLink applications, the system adopts but is not limited to ultra-low latency (first category) data multi-node concurrent transmission and NearLink low power access (SLE) technology. When the smart terminal is composed of a consumer-grade smart phone or tablet, notebook, or desktop computer with NearLink technology functions (the second NearLink module), data collection, processing, caching, control, etc. can be jointly implemented by the hardware module and App (Application). The storage and output module of the smart terminal can realize the local ECG display, printing, monitoring required in different scenarios through App software and the Internet or transmits it to specialists for diagnosis in real time through the Internet and can upload the measured electrocardiogram report data to the cloud for remote diagnosis and big data analysis.

The foregoing description of the exemplary embodiments of the invention has been presented only for the purposes of illustration and description, and is not intended to be exhaustive or to limit the invention to the precise forms disclosed. Many modifications and variations are possible in light of the above teaching.

The embodiments were chosen and described to explain the principles of the invention and their practical application to enable others skilled in the art to utilize the invention and various embodiments and with various modifications suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

What is claimed is:

1. A wireless electrocardiogram (ECG) system for medical diagnosis, comprising:
a smart terminal;
a plurality of lead front ends, each lead front end being coupled with a corresponding body surface electrode configured to be attached to a chest of a subject for collecting ECG/biopotential signals; and
a bidirectional wireless communication channel established between each lead front end and the smart terminal for independent bidirectional information transmission;
wherein the wireless ECG system is configured to have a simulation testing mode and an actual measurement mode, such that close-loop adaptive ECG information collection and process are achieved by switching between the simulation testing mode and the actual measurement mode online in real time;
wherein when in the simulation testing mode, the smart terminal first acts as a signal source to synchronously send a calibration/digitized ECG signal as an input reference signal to each lead front end through the bidirectional wireless communication channel, and the information obtained by the lead front end for detecting the reference signal is transmitted back to the smart terminal through the bidirectional wireless communication channel, and the smart terminal then compares and calibrates the reference signal detected by each lead front end with the calibration/digitized ECG signal originally sent and generates the test response curve of each channel;
wherein when in the actual measurement mode, the ECG/biopotential signal collected by each body surface electrode is pre-processed by the lead front end connected to one by one, and then uploaded to the smart terminal through the bidirectional wireless communication channel, and the smart terminal corrects the ECG data actually measured by each lead in real time based on the response curve to obtain an ECG report and output it.

2. The wireless ECG system of claim 1, wherein the plurality of lead front ends comprises seven or more lead front ends, or ten or more lead front ends.

3. The wireless ECG system of claim 1, wherein each lead front end comprises a first NearLink module, and the smart terminal comprises a second NearLink module; the first NearLink module and the second NearLink module perform short-distance wireless communications through the NearLink mode, and the bidirectional information transmission is realized between each lead front end and the smart terminal through the first NearLink module and the second NearLink module, respectively.

4. The wireless ECG system of claim 1, wherein the smart terminal comprises a signal generation module and a data processing module, the signal generation module is configured to generate a calibration/digitized ECG signal and an adaptive control signal, and transmit it downlink to each lead front end as an input reference signal; the data processing module is configured to compare the information detected by the reference signal wirelessly uplinked back from each lead front end with the original transmitted signal and generate a test response curve of each lead front end; and the measured ECG data of each lead is corrected in real time according to the response curve.

5. The wireless ECG system of claim 1, wherein the smart terminal comprises a time base and control module, the time base and control module is configured to generate a high-precision time base signal and synchronously control the signal sampling and information wireless transmission of each lead front end in a concurrent manner, and the internal calibration of each lead front end and the input switching of the ECG measurement between the simulation testing mode and the actual measurement mode.

6. The wireless ECG system of claim 1, wherein the smart terminal comprises a storage and output module, the storage and output module is connectable to the cloud through the Internet and is configured to cache and normalize the ECG data and then store it locally and/or upload it to the cloud, and output the ECG data in the form of an ECG report.

7. The wireless ECG system of claim 1, wherein each lead front end comprises an input circuit module and a signal processing and control module, the input circuit module pre-processes the ECG/biopotential signals collected by the body surface electrodes, and the signal processing and control module uploads the pre-processed ECG/biopotential signals through the wireless communication channel under the control of the smart terminal; and completes the input and output of the reference signal for the simulation testing mode transmitted by the smart terminal.

8. The wireless ECG system of claim 1, further comprising a storage bin, wherein the storage bin is operably connected to the smart terminal via a C-Type interface and/or NearLink wireless communication channels; the storage bin is configured to store the lead front ends and charge the lead front ends; before each ECG test, the storage bin cooperates with the smart terminal to perform initialization calibration, calibration and display of a charging level of each lead front end.

9. The wireless ECG system of claim 1, further comprising one or more expandable lead front end, wherein a bidirectional wireless communication channel is established between the expandable lead front end and the smart terminal, and the expandable lead front end is configured to detect other physiological information of the subject.

10. The wireless ECG system of claim 1, wherein the communication protocol used by the wireless communication channel comprises NearLink, WiFi, Bluetooth, Radio Frequency Identification (RFID), Near Field Communication (NFC), ZigBee, Ultra-Wideband (UWB), Electric Potential Sensing Communication (EPSComm), ANT, Z-Wave, Thread and infrared technology, and any combination thereof.

11. The wireless ECG system of claim 1, wherein each lead front end is a wireless wearable member, and logo and color of the wireless wearable member of each lead front end are determined according to electrode identifier and color code specified by national standards.

12. The wireless ECG system of claim 1, further comprising a positioning ruler personalized/customized specifically for the subject, wherein the positioning ruler is formed of a quick-curing polymeric/shape memory medical material on site of the chest of the subject to conform to the curve shape of the chest of the subject.

13. The wireless ECG system of claim 12, wherein the instantly solidified medical material comprises a low-temperature thermoplastic tape, a thermoplastic splinting material, a food-grade dental mold silicone, or other materials that meet human safety standards.

14. The wireless ECG system of claim 12, wherein the positioning ruler has two ends marked with exact positions (and the third positioning point, if necessary) relative to body markers of the subject, and a plurality of holes formed therebetween for placing the body surface electrodes on the chest of the subject.

15. The wireless ECG system of claim 14, wherein the holes of the positioning ruler is personalized/customized such that when the positioning ruler is placed on the chest of the subject with the ends aligned with the body markers, the positions of the holes coincide respectively with the positions of the body surface electrodes to be placed.

16. The wireless ECG system of claim 15, wherein in operation, the body surface electrodes are placed on the chest at the positions of the holes of the positioning ruler.
